(19) United States
(12) Patent Application Publication
(10) Pub. No.: US 2026/0260725 A1
(43) Pub. Date: Sep. 3, 2026

(54) SYSTEMS AND METHODS FOR OPTIMIZING WORKFLOW OF REPORTS BASED ON DIAGNOSTIC IMAGES

(71) Applicant: Radpair Corp., Knoxville, TN (US)

(72) Inventors: Avez Rizvi, Austin, TX (US); Koorosh Zarezadeh, Nanaimo (CA)

(21) Appl. No.: 19/163,808

(22) PCT Filed: Mar. 11, 2024
(86) PCT No.: PCT/US2024/019469

§ 371 Date: Sep. 9, 2025

Related U.S. Application Data
(60) Provisional application No. 63/451,165, filed on Mar. 9, 2023.

(51) Int. Cl. G16H 15/00; G06F 40/166; G10L 15/26; G16H 30/20; G16H 50/20

(52) U.S. Cl. CPC G16H 15/00; G06F 40/166; G10L 15/26; G16H 30/20; G16H 50/20

(57) ABSTRACT

The present technology relates to methods and systems for streamlining the process of diagnostic imaging; in particular, that introduce parallel, rather than sequential, reading, accomplished by generative artificial intelligence (A.I) including large language models (LLMs). The methods and systems herein can also include additional features, such as a dynamic user interface, facilitation of real-time editing, text formatting, gamification elements or features, and an interoperability module that is configured to adopt a variety of healthcare standards. The technology constitutes a significant overhaul on traditional radiology and healthcare workflow processes.

### CROSS-REFERENCE TO RELATED APPLICATIONS

This application claims the benefit of U.S. Provisional Application No. 60/451,165 filed Mar. 9, 2024, which is hereby incorporated by reference in its entirety.

### BACKGROUND

The present technology relates to the field of radiology, and in particular, to methods and systems for streamlining the process of diagnostic imaging.

Historically, in known radiological process, photographic images from a patient were captured on films by a radiologist or another medical professional, such as a radiology technician. These images were then developed and then “hung” on lightboxes for the radiologist's review. These lightbox films were typically reviewed by the radiologist in conjunction with a transcriptor or transcriptionist (another human who would write down the notes as verbally uttered by the radiologist) or recorder (a machine such as a tape recorder, having a microphone that the radiologist could speak into). The radiologist would then move onto the next case. In this form of reporting, the actual report was not generated at the time of diagnosis; instead, the radiologist would review all of the cases and call out findings in a verbal, natural language, narrative form. Medical transcriptors would write down the radiologist's words, or obtain and listen to the recordings. They would thereafter translate the narrative findings into a professional report, abstracting the word processing and non-narrative language from the radiologist's words.

Unfortunately, this process increased turn-around times, as the reports would not be processed in real time, and there were delays in developing films, obtaining the tape recordings, and translating the language.

Over the past decades, advancements have been made in this process. Specifically, most radiological images are currently captured digitally rather than on film. This has sped up the process by making it possible for a healthcare technician to save the images digitally at the time they are captured from the patient, and for the radiologist to then access the images on a computer screen, without the delay caused by developing film. Further, medical transcription technology has advanced, as recordings of the radiologist's speech can also now be done digitally, and accessed from any point without the need to transfer a physical tape recorder from the radiologist to the transcriptor. Voice recognition technology has also made it possible for a radiologist's language to be contemporaneously captured in digital files, without the need for a human to take notes.

Despite these advancements, current technology still presents inefficiencies and delays. Current voice recognition technology does permit real-time transcription of a radiologist's spoken language into written text. However, this approach is still limited in that: (1) it shifts the burden of report creation to radiologists; and (2) the current systems are limited to providing text output exactly as the words are spoken. Thus, all punctuation such as periods and new lines must be precisely dictated. Also, in the flow of speaking, humans naturally pause or say, “umm” or other non-language phrases (this is the essence of free-form and natural speech). With current voice recognition technologies, the human who is speaking must be extremely precise, with no variability in speech, to achieve the desired results. The necessity of focusing in this matter leads to greater inefficiencies in the process.

There are further limitations to workflow for looking at images and diagnosing. Traditionally, the steps of: (a) looking at images to diagnose; and (b) creating reports; have been done sequentially by healthcare professionals. As an example, if a radiologist has a typical worklist with several cases, he must open each one separately, look at each set of images, and then generate a report for each case. His mind will typically be split between paying attention to the images while simultaneously speaking to the voice recognition in a very precise manner in order to avoid mistakes. Mistakes, which invariably occur, then require the radiologist to context switch between the images and the report to fix the errors. Once accomplished, the radiologist signs off on the report and then moves onto the next case in the worklist. This sequential approach has been the current state of radiology reporting for several decades.

Therefore in view of increasing demands on the technical expertise of radiologists and other physicians, and a desire for efficiency and faster turnarounds for diagnostic tests, a need continues to exist for methods and systems that increase the efficiency of the ways in which radiologists review patient data, diagnose medical conditions, and provide reports that satisfy industry medical standards.

### BRIEF SUMMARY

The present technology constitutes an overhaul on traditional radiology workflow by introducing parallel (rather than sequential) reading plus reporting accomplished by generative artificial intelligence (A.I.).

In certain embodiments, the present technology is directed to a method for generating a report based on a healthcare professional's review of a diagnostic image, the method comprising the steps of:
(a) scanning the body of the patient with an imaging apparatus;
(b) generating an image based on the scan;
(c) storing the image in a server along with additional patent data, wherein the image and the patient data are associated with each other in the server;
(d) obtaining a diagnosis of a medical condition from a medical professional, wherein the diagnosis is in the form of speech in narrative form from the healthcare professional; and
(e) translating the speech in narrative form into speech suitable for a report that satisfies an industry standard, through the application of machine learning to generate a report in written form, wherein the report also contains the additional patient data.

In various embodiments, steps (a) through (d) described above are accomplished in parallel, or contemporaneously, over 2 or more diagnostic images; or using the same computer processor.

In certain embodiments, the present technology is directed to a system for providing a report based on a healthcare professional's diagnosis, the system comprising:
(a) a user interface that displays a diagnostic image obtained from the body of a patient;
(b) a microphone configured to capture audio data in the form of speech in narrative form from the healthcare professional based on the healthcare professional's visual observation and diagnosis of the diagnostic image; and
(c) a computer processor configured to: (i) provide a transcription of the speech in voice recognition (VR) form based on the transcription speech in narrative form; (ii) associate the transcription of the speech in VR form with identifying information of the patient already stored in the system; and (iii) generate the report including the transcription of the speech in VR form and identifying information of the patient;
wherein the computer system is configured to conduct steps (a) through (c) for more than one patient in parallel or contemporaneously, while interacting with a single healthcare provider.

In certain embodiments, the present technology is directed to a method of diagnosing a medical condition in a patient, the method comprising the steps of:
(a) scanning the body of the patient with an imaging apparatus;
(b) generating an image based on the scan;
(c) storing the image in a server along with additional patent data, wherein the image and the patient data are associated with each other in the server;
(d) obtaining a diagnosis of a medical condition from a medical professional, wherein the diagnosis is in the form of speech in narrative form from the healthcare professional; and
(e) translating the speech in narrative form into a form suitable for a final report according to an industry standard, through the application of machine learning to generate a report in written form, wherein the report also contains the additional patient data.

### BRIEF DESCRIPTION OF THE DRAWINGS

FIG. 1 shows a typical radiology workflow for a known method or system for providing a medical diagnosis.

FIG. 2 shows a system according to an embodiment of the present technology.

### DETAILED DESCRIPTION

As used herein, all singular terms refer to both singular and plural values. That is, “a” or “an” or “the” all mean “one or more.” The term “or” as used herein means any one or more of the alternatives, including all of the alternatives.

Throughout the present disclosure, when described in sequential words (for example, using “then” or “next”), such description is not limiting to the described steps in the particular order set forth, but also includes embodiments wherein the steps are presented in any order.

As used herein, “medical condition” means any state of health, whether ill or healthy, in a patient. This can include diseases or disorders. As used herein, “disease” means any deviation from, or interruption of, the normal structure or function of any part of the body. As used herein, “disorder” means any abnormal condition of the body.

As used herein, “radiology” means the medical specialty that involves the use of medical imaging to diagnose medical conditions, as well as guiding the treatment and treating such conditions, including diseases and disorders. As used herein, “medical imaging” means the collection of any visual data from the body of a patient, using an imaging apparatus. As used herein, “imaging apparatus” means any portion of any machinery that can obtain visual data, such as an image, from the body of a patient.

Generally, in radiology, numerous radiological procedures can be employed; including computerized tomography (CT or CAT) scan; magnetic resonance imaging (MRI), X-ray, ultrasound (US) and the like. The machinery employed in such procedures is “imaging apparatus” for the purposes of the present disclosure.

As used herein, “radiologist” means a medical doctor (physician) who specializes in radiology. In certain embodiments herein, the review of the patient data (including images) can be performed by a radiologist, but can also be performed by any healthcare professional who is qualified to do so. Throughout the present disclosure, when referring to a radiologist, the embodiments herein contemplate any healthcare professional who is qualified and permitted to perform the task of a radiologist.

As used herein, “medical professional,” “health professional” or “healthcare professional” means any person licensed or certified to provide health care services to people, including but not limited to physicians, physician assistants, nurses, nurse assistants, emergency medical personnel, and technicians (for example, radiology technicians or sonographers). As used herein, “user” or “viewer” means anyone who can access a method or system herein in order to obtain data, including patient images or any other information, whether a human (for example, any healthcare professional or a patient or someone authorized by the patient) or computer.

As used herein, “radiology workflow” means any system or method in the field of radiology that involves scanning the body of a patient and then using the information obtained to diagnose a medical condition such as a disease or disorder, including the steps included in such a system or method.

As used herein, “narrative form” or “free form” means any language having a human source that constitutes the combination of techniques chosen by a writer or speaker in telling a particular story, and that is spoken with a natural structure and style. As used herein, “voice recognition form” or “VR form” means a form created by a machine or program based on its receipt and interpretation of spoken commands from a human voice. VR form is generated at least in part through biometric technologies such as speech recognition or machine translation.

As used herein, “artificial intelligence” or “A.I.” means the simulation of human intelligence processes by machines, in particular, computer systems, including the ability to learn, read, write, create, and analyze. As used herein, “generative A.I.” means A.I. that is capable of generative text, images or other data using generative models, which learn the patterns and structure of their input training data and then generate new data that has similar characteristics. As used herein, “large language models” or LLMs are a type of generative A.I. that can be multi-modal (as used in images, text or video).

As used herein, “industry standard” means a standard that is widely accepted and adhered to within the industry, e.g., a standard for reporting that is set by the American College of Radiology, Radiological Society of North America, American College of Radiology, or similar professional organization that governs reporting standards.

As used herein, “parallel” and “contemporaneous” are used interchangeably, and mean in the same period of time, including in the same medical procedure, but not necessarily exactly simultaneously. Discussions herein about reviewing patient data such as diagnostic images “in parallel” refer to a healthcare professional's ability, through the methods and systems herein, to review and report more than one case at about the same time, in contrast with sequential reviewing, which requires that the healthcare professional substantially complete the reviewing and reporting steps of one case before starting a subsequent case.

As used herein, “immediate access” means the ability to access information contemporaneously with another action, in various embodiments, within less than 5 minutes, within less than 3 minutes, or within less than 1 minute. As used herein, “quick access” means the ability to access information promptly, in various embodiments, within less than 4 hours, within less than 1 hour, within less than 30 minutes, or within less than 10 minutes. As used herein, “real-time” and “on-the-fly” are used interchangeably, and mean a user's ability to accomplish a task as a necessary element of that task is being communicated to the user, without the need for the user to pause to wait for subsequent necessary elements of the task to be communicated.

### Traditional Radiology Digital Workflow

Traditionally, a radiology workflow involves two broad components or steps: (1) Image Acquisition; and (2) Reporting.

### (1) Image Acquisition

Image Acquisition is typically obtained through a radiology modality or process, such as CT, MRI, XR (X-ray) or US. This can involve the orchestration of various software systems.

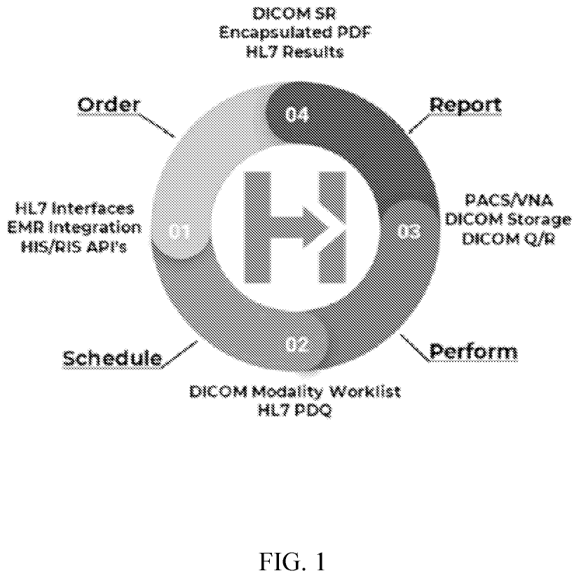

Orchestration can begin at the level of order entry, e.g., within an Electronic Medical Record (EMR). Digital Imaging and Communications in Medicine (DICOM) is an international, nonproprietary standard that specifies protocols for exchanging medical images and related data in healthcare systems. Using the language of DICOM, the capturing of a medical image is an “acquisition” and imaging equipment is an “acquisition device.” Thus, in a typical radiology workflow, an order inputted by a medical professional is translated into a scheduled event within a DICOM Modality Worklist (DMW), for example, as shown FIG. 1.

HL7 (Health Level Seven) is a set of international standards that are used to communicate and exchange information between different healthcare systems, for example, between software platforms in the healthcare industry.

In a typical radiology workflow, image acquisition occurs at the time of the procedure, and the acquired images are transferred as DICOM files to an archival system. A Picture Archiving and Communication System (PACS) can retrieve the images (which can be locally cached) to display to a viewer (such as a computer screen). The details of the acquisition can vary depending on the systems used.

### (2) Reporting

Reporting is the mechanism by which a radiologist can review images and create a report. The workflow for reporting typically includes additional software systems for displaying the worklist of studies to be reported. These systems can include Radiology Information Systems (RIS), PACs, or Voice Recognition (VR) systems. These can display the worklist and can be utilized dependent on which schema is preferred by the institution.

But regardless of the schema used, the traditional workflow shares a common style of sequential reporting—that is, a radiologist picks up a single study from the worklist, reviews the study, reports the study, and then picks the next study to repeat the process.

Thus, a summary example of a typical known radiology workflow is as follows:
1. Study Order: The ordering physician puts a study order for a CT scan into the EMR system. The order includes patient information, study details, and any relevant clinical history.
2. HL7 Interface: The EMR system sends the study order information to a Radiology Information System (RIS) through an HL7 interface. The HL7 interface uses a standardized format to exchange health information between the systems.
3. Scheduling: The RIS schedules the CT exam for the patient and sends the appointment information back to the EMR through the HL7 interface.
4. Image Acquisition: During the CT exam, the patient is positioned and the CT images are acquired using a CT scanner. The CT scanner generates DICOM images and sends them to a Picture Archiving and Communication System (PACS).
5. DICOM Workflow: The PACS stores the DICOM images and provides access to them for the radiologist and any other medical professionals. The PACS also sends the images to the RIS through a DICOM interface.
6. Radiologist Review: The radiologist reviews the CT images in the PACS, writes a report, and saves it in the RIS.
7. Report Distribution: The RIS sends the report back to the ordering physician and any other relevant medical professionals required through the HL7 interface. The report is also stored in the PACS for future reference.
8. Final Report: The final report is available in the EMR for the ordering physician to review and use for patient care.

Traditional systems were created and integrated with the above-described form of sequential reading, and are nearly ubiquitous among radiologists worldwide. In the most traditional processes, as described above, radiology workflow involved developing films based on scanned information from the patient's body. However, over the past several decades, systems optimization, including caching of images and user interface/user experience (UI/UX) options within all PACS, RIS, and VR systems have adopted this form of workflow, ever since digital imaging became the mainstay of reporting. Modifications of UI/UX have occurred within the industry; however, these have focused on incremental changes, with the core of workflow remaining sequential and stale.

Further, traditional radiology reporting systems are often static and offer limited interactivity. They commonly rely on text-based or Rich Text Format (RTF) outputs. These static interfaces have not kept pace with advancements in User Interface (UI) and User Experience (UX) design. As a result, they lack features that could otherwise make reporting more efficient and user-friendly.

VR system usage has replaced historical transcription services performed by human transcriptors, to improve turnaround times for reporting. In the current landscape, radiologists themselves are responsible for word processing and generation of accurate reports in real time. Dictation systems take in radiology-specific lexicon, used by the radiologists, and convert them to text within a software system interface. Additional tools such as templates and macros can be utilized, but also require additional bandwidth and processing by the individual radiologists.

A downside of the adoption of VR systems is that this has changed the narrative and free form of calling out findings, into a lexicon-specific and systematic style of reporting. Thus, the radiologist often must use an unnatural form of dialectic to optimize for a VR system's requirements.

The following are examples of how reporting has changed through the years, with the advent of computerized transcription services such as VR:
Narrative Form: “Ok, so there's a simple cyst in the right kidney and it looks to be measuring about 10 Hounsfield units. It also has some mild hydronephrosis and an obstructing stone in the left UVJ measuring about 3 mm. Some mild degenerative changes are there in the lumbar spine. That's about it.”
VR Specific Form: “There is a simple cyst in the right kidney, comma, and it measures approximately 10 HU, period. There is mild hydronephrosis secondary to an obstructing 3 mm calculus in the left UVJ, period. There are mild degenerative changes along the lumbar spine, period.”

In the VR-specific language above, the language is no longer narrative, natural and free form, but rather must be robotic, and requires the radiologist to recite explicitly all punctuation out loud. This is necessary for the text to be translated exactly in a professional manner for reporting. Even after such form of reporting is converted to text, the radiologist still must ensure that the formatting and location of descriptions, including pertinent negatives, are present within the report. It is not difficult to see that radiologists do often struggle with adapting to such a style. This can lead to further delays and inefficiencies.

Despite the numerous challenges with using unnatural forms of speaking with VR and associated loss of efficiency (and increased likelihood of errors), the technology and workflows associated with VR have been adopted nearly universally worldwide.

### A New Workflow: Digital Parallel Reporting

In certain embodiments herein, new workflow methods and systems have been developed, providing a solution to the disadvantages of known processes.

In certain embodiments, the present technology is directed to a method or system that comprises a hybrid software model of image reporting in real time, that constitutes an improvement over known systems.

In certain embodiments, a reporting system herein can utilize integrated chatbots, such as OpenAI's ChatGPT3 in place of a human transcriptionist. Parallel reporting can occur through a new system that is capable of launching multiple cases simultaneously, instead of sequentially, for purposes of reporting. In certain embodiments, a method or system herein harnesses the capabilities of advanced generative A.I. technologies, through the deployment of large language models (LLMs) (including but not limited to OpenAI's GPT-3), to accurately interpret and transform natural, free-form speech from the radiologist and radiological data into a comprehensive report. Additionally, the methods and systems herein can, in certain embodiments, comprise one or more parallel processing features, to permit the substantially simultaneous caching and handling of multiple reports, facilitating a more efficient, scalable reporting workflow by allowing for the substantially concurrent analysis of multiple cases, moving beyond limitations of linear and sequential processing.

In certain embodiments, a method or system herein combines the following 3 components:
A. Parallel Viewing Software—In certain embodiments, a method or system herein can include one or more of on-premises or cloud-based technology that can locally cache or edge/cloud compute, thereby immediately displaying multiple fully loaded image studies contemporaneously in an easily consumable format, e.g., on one or more computer screens. This can, in certain embodiments, include tabbed windows, or other UX methods to differentiate studies. A functional UI/UX can be aimed at image interpretation in each study, in contrast to comparison-only displays, as is the case with current multi-study viewing systems.
B. Voice-Enabled Chatbot Integration—In certain embodiments, a method or system herein can integrate a chatbot with additional patient data, for example, necessary study type and demographic information that is algorithmically obtained from DICOM header or HL7 information. For example, the sex of the patient, age, other identifying information of the patient (e.g., address, next of kin, primary care physician), or type of study can be known to the chatbot, and used to generate the report. In certain embodiments, narrative and natural (unlike VR) call-out of findings by the healthcare professional, for example, by speaking into a microphone or any other device that can record the healthcare professional's voice, can be combined with algorithmically-obtained information, as well as coded rules, to generate a report automatically.
C. Integrated Report Generation Window—In certain embodiments, an auto-generated report can be displayed in a separate window, and can be locked by default to the viewer tab (UX) within the parallel viewing software of a method or system herein. This can prevent errors of reporting, and can also serve to associate the report with the study, in the event that the radiologist needs to review. In certain embodiments, a method or system herein can be configured such that the radiologist can review the finalized report, edit if necessary, and sign the report. In certain embodiments, an integrated report window can also include other options, e.g., the option to regenerate a report by the radiologist's calling out new information. For example, a command such as, “Please add the right kidney has a 4 mm interpolar renal calculus” can be ingested by the software, and a report regenerated with the updated information.

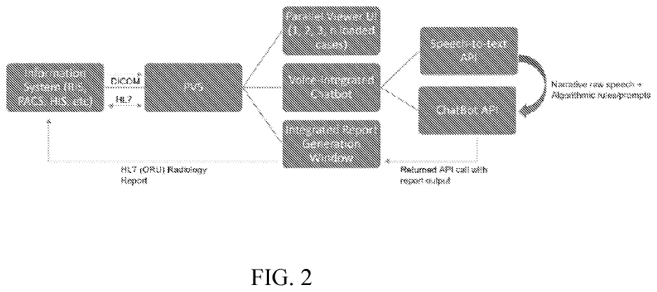

FIG. 2 shows an embodiment of a method or system contemplated by an embodiment herein.

The above 3 components are discussed in further detail below:

### A. Parallel Viewing Software

In certain embodiments, a method or system herein includes Parallel Viewing Software (PVS) that incorporates the tools necessary to review cases by radiologists and other diagnosticians. As used herein, “parallel” means contemporaneous, in that every feature need not be performed at exactly the same time, but can be within the same medical procedure, and is sufficient for the medical professional to appreciate the ease of being able to access the information as part of the review and analysis of the results and the determination of a professional diagnosis.

In certain embodiments, a method or system herein comprises a step wherein the healthcare professional reviews diagnostic data from the patient, including a diagnostic image, for example, one obtained through ultrasound, X-ray, MRI or any other machine that can generate images for review by a healthcare professional such as a radiologist. In certain embodiments, the diagnostic image is a DICOM (Digital Imaging and Communications in Medicine) image file (also referred to herein as a DICOM object), which can have DICOM tags or protocols.

In certain embodiments, a method or system herein can at least substantially fully integrate with the PVS, and can include RIS, PACS, and other DICOM server applications. The following are exemplary steps, one or more of which can be included in a method or system of an embodiment herein:
1. DICOM Query/Retrieve (Q/R): In certain embodiments, a method or system herein provides for PVS that can integrate with a PACS through the DICOM Q/R protocol. For example, the PVS can send a query to the PACS server to retrieve one or more particular images or studies based on parameters such as patient name, ID, other identifying information, or study date. The PACS server can send the requested image or study to the PVS in the DICOM format.
2. HL7 Interface: In certain embodiments, a PACS system can integrate with PVS using one or more HL7 interfaces. In such embodiments, a PACS server can send a message to the viewer (for example, a radiologist or another medical provider) containing information about one or more new studies or images, and the viewer can retrieve the new study or image from the PACS using DICOM Q/R.
3. Web-Based Access: In certain embodiments, a PACS system herein can provide web-based access to any image stored in the system. In such a case, the PVS can access the PACS through a web browser, and a viewer can view the images directly without the need for a separate computer application.
4. Vendor-Neutral Archive (VNA): In certain embodiments, a method or system herein comprises a system that can store patient data, including medical images and related information, in a standardized format (a VNA), thus making it easier to share and integrate such data with other systems. In such embodiments, PVS can integrate with a VNA to retrieve the data from multiple PACS systems or other sources.
5. Cloud-Based Access: In certain embodiments, a method or system herein comprises a cloud-based PACS system, which can allow users to access and view data such as images from any location with an internet connection. For example, PVS can integrate with a cloud-based system to permit the user to retrieve and view images from the system.

In certain embodiments, regardless of the level of integration among systems, the PVS can be configured to exhibit certain unique additional functions not currently available. These can include one or more of the following:
6. The ability to open multiple cases in parallel across UI, that is specifically intuitive for interpreting cases.
   a. In certain embodiments, this can include access to all of the tools and information available to interpret per UI per case. For example, tabbed windows, each serving as a standalone and fully functional viewer for a loaded case—that is, each tab can function the same way as a traditional serial viewer could function, but contemporaneously rather than in series (e.g., multiple serial viewers built into a single system).
7. In certain embodiments, intelligent caching and sorting of images is available, that can allow more than one study to be loaded at once, without resulting in latency or lag issues. This can include, but is not limited to, locally caching select data (e.g., select images), or processing at least substantially all caching in the cloud (dependent upon cloud v. on-premise setup). For example,
   a. Server-side caching: This can involve storing images on the server in memory or on disk. This can reduce the load on the server and improve the response time when retrieving images or other data. Server-side caching can also employ techniques such as content delivery networks (CDNs) to distribute images across multiple servers, further improving performance and scalability.
   b. Preloading: This can involve loading images in the background before they are needed. This can improve the response time when the user requests the images, thus reducing the load on the server. In certain embodiments, preloading can be combined with caching to improve performance further.
8. In certain embodiments, a method or system herein provides for optimized UX features, which can allow for parallel reading. For example,
   a. In certain embodiments, error prevention methods are present, which can tie the UI viewer windows to patient reporting, to prevent wrong patient reporting.
   b. In certain embodiments, a method or system herein includes intelligent prioritization of case loading. For example, a method or system herein can provide integration rules with RIS/PAS DICOM, e.g., a modality worklist, wherein priority is established to pull up cases based on their relative urgency; e.g.:
      i. Modality worklist priority tag: The DICOM standard defines a priority tag (0074,1200) that can be used to assign a priority level to each worklist item. The priority levels range from 0 (lowest) to 2 (highest), with level 2 indicating a critical or urgent case. In certain embodiments, a method or system herein can assign a priority level to each worklist item (or the ordering physician can do this), such that the PVS can flag critical cases and prioritize them accordingly.
      ii. Custom worklist fields: In addition to standard DICOM worklist fields, in certain embodiments, custom fields can be added to a worklist to provide additional information about each exam. For example, a custom field could be used to indicate the urgency or criticality of a particular exam. PVS can then use this information to prioritize the exams and flag critical cases.
      iii. Worklist filters: In certain embodiments, one or more worklist filters can be used to sort and prioritize the worklist items based on particular criteria. For example, in certain embodiments, a filter can be created to show only exams with a priority level of 2, or exams with a particular diagnosis or clinical indication. This can help PVS to prioritize critical cases and ensure that they are performed as soon as possible.
      iv. Worklist annotations: In certain embodiments, one or more annotations can be added to worklist items to provide additional information about each exam. For example, in certain embodiments, a filter can be added to indicate that a particular case is critical or urgent. PVS can then use this information to prioritize the exams and flag critical cases.
   c. In certain embodiments, a method or system herein can provide for at least substantially continuous loading of a new set of cases once interpretation is finalized. For example, if three tabs or opened, each with an individual set of study images for one patient, and tab 1 is interpreted and completed first, followed by tab 2 and then tab 3, then once tab 1 is finalized (i.e., the radiologist signs off), it will immediately load the next prioritized case, while the radiologist moves on to interpreting tab 2 and tab 3. This cycle can repeat as the cases are reviewed.

### B. Voice-Enabled Generative A.I., LLM-Based Integration

In certain embodiments, PVS can be fully agnostic to chatbot application programming interfaces (APIs) such as OpenAI's ChatGPT or Google's Gemini (formerly known as BARD), Anthropic's Claude LLM or Meta's Llama2 open source model, and other models, and can work with any available large language model. Additionally, in certain embodiments, a voice-enabled, speech-to-text function can also be fully agnostic to available speech-to-text APIs such as IBM Watson, Amazon Transcribe, Google Cloud Speech-to-Text, or any other API.

In certain embodiments, a PVS as discussed herein can integrate a voice-enabled generative A.I., LLM-based UI and UX (e.g., a chatbot) as an applet running within the system, that can perform one or more of the following functions:
1. Receives voice input from the user and uses the speech-to-text API for near real-time translation to text.
2. Provides for translated text to be viewable in a separate UI to allow a user to error check and edit any output in, for example, a word processing tool.
3. Provides for the ability to send a text message, for example, via generative A.I., LLM-based application programming interface (API) for translation of text to a radiology report, with, optionally, one or more of the following unique algorithmic techniques:
   a. Automatically parse out DICOM header information relevant to creating the report, e.g., “study type,” “contrast/bolus agent,” “study instance UID (for comparison studies)” or other relevant tags to pass into preset fields along with the speech-to-text data to prompt the generative A.I., LLM-based API to process the report correctly.
   b. Unique algorithmic prompting to allow the generative A.I., LLM-based API to access a specific set of instructions to produce standardized, professional radiology reports, including prompts on, for example: the templating of reports; the organization of pertinent positive or negative findings; the impression or conclusion of reports; recommendations or guidelines to use for the reports; particular formatting for the reports; customized statements for the reports.
4. Provides for a rules engine to limit responses to the radiology report generation. For example, in certain embodiments, entering information unrelated to the radiology report will not result in a response, or will initiate an error sequence. This can include, e.g., rules for adding a specific custom prompt or request to the API call outside of narrative text—for example, a prompt to use a different set of guidelines or recommendation criteria.

### C. Integrated Report Generation Window

In certain embodiments, a PVS herein can include an integrated report generation window, for example, a word processing tool that can receive the returned API call response from the generative A.I., LLM-based API in the form of a report. In certain embodiments, one or more of the previously-mentioned can occur algorithmically. In various embodiments, the tool can include, but is not limited to:
1. A word processing tool that can include one or more of the following: the ability to edit the report, at least basic panel formatting options (e.g., tools for numbering, bulleting, margins, text effects such as bolding and the like).
2. The ability to undo the returned API call data, restoring it to voice narrative input from the user.
3. The ability to continue to add narrative voice-enabled text to the data.
4. The ability to resend the API call and receive an updated response in reply.
5. The ability to sign the report. This can, in certain embodiments, pass the API call response to an HL7 ORU (H17 Observation Result) message carrying the radiology report. The ORU message can be sent to the hospital information system (HIS) or electronic medical record (EMR) system using a secure communication protocol such as the internet protocol suite TCP/IP or HL7 over HTTPS (hypertext transfer protocol secure).
6. The ability to lock the report generation window, or to associate it with the PVS case UI, to prevent errors in reporting. In certain embodiments, this can be done through certain UI methods, including but not limited to: color coding, tying universally unique identification (UUID) information, or tracking tabs or the like.

In certain embodiments, newer methods of image retrieval and transfer are available and contemplated within the embodiments herein (for example, cloud-based systems).

### Web-Based Fine-Tuning and Gamification Interface for Personalized Radiology AI Report Generation

In certain embodiments, a system herein amalgamates two or more of the following: one or more gamification elements or features, real-time AI model fine-tuning, and a web-based interface; to provide radiologists with a platform where they can not only generate reports but also incrementally train and personalize the underlying AI model based on their unique preferences and feedback.

Current AI-powered reporting systems are generally “one size fits all.” Any customizations (if even available) tend to require a hefty computational load, intricate processes, and the expertise of data scientists. An advantage of the present technology is the ability for healthcare professionals to personalize an AI model seamlessly, and to be actively engaged in its evolution.

Thus, in certain embodiments, a method or system herein provides a web-based interface that has high accessibility, in that it can facilitate easy access from multiple devices. In certain embodiments, profile features include any of the following: allowing users to monitor their usage metrics, progression milestones, accumulated rewards, and a model that includes user (for example, a single click or user interaction) to access required information.

In certain embodiments, a method or system herein also includes a gamification system that can provide any of the following gamification elements or features: engagement through rewards, wherein a user can accumulate “points” through interactions, e.g., by dictating findings, generating and finalizing reports (thus incentivizing efficiency on the part of the healthcare workers who use the system); provided visual progression, e.g., a visual dashboard displaying an overview of the user's progression, keeping users abreast of their journey to a goal such as a reward or a model upgrade; or other incentives for user empowerment, such as options at specific milestones (e.g., an upgrade of the A.I. model), which can, in certain embodiments, be in the form of an intuitive one-button mechanism.

In certain embodiments, a method or system herein can also accomplish one or more of the following: (1) Behind the scenes integration: For example, upon activating the upgrade, the system can then be refining the A.I. model, tailored to the user's historical inputs and preferences, without the need for the user to dive into the specifics or technicalities. (2) Advanced data processing: including, for example, tailored training, wherein a system herein can process user-specific data, employing predefined LoRA (low-rank adaptation or large language models) ranks to ensure that the A.I. model fine-tuning aligns meticulously with individual user behaviors.

In certain embodiments, a method or system herein can achieve the streamlining of computational processes, in contrast with traditional model training, which demands significant computational resources and time (in particular with large datasets). Advantages of the embodiments herein include: (1) Incremental Learning: Instead of periodic extensive training sessions, a model herein can undergo frequent, smaller fine-tuning sessions based on user-specific data. (2) Reduced load: As the system continually refines pre-existing models rather than training from scratch, it significantly conserves computational resources. (3) Rapid deployment: In certain embodiments, a streamlined fine-tuning allows for quicker model deployments, ensuring users benefit from enhanced versions in near-real-time.

Thus, in various embodiments, the present technology is directed to: a radiology reporting system that integrates one or more of: a web-based interface, a gamification element, or real-time A.I. model fine-tuning (e.g., A.I. model refinement or fine-tuning of one or more elements of the report).

[symbol] , that can be specifically tailored for personalization by an individual healthcare provider.

And optionally, wherein any of the following are true of the system:
the A.I. model fine-tuning operates in an incremental manner, conserving computational resources and enabling rapid deployment;
the system employs gamified elements to visually showcase user progression towards achieving milestones linked to model upgrades;
the system's fine-tuning process is automated, yet personalized based on the specific user's interactions and feedback;
the system features an intuitive dashboard providing an overview of the user's progression, accumulated points, and available model upgrades;
the system is optimized for resource efficiency, ensuring computational and storage resources are judiciously utilized for model refinements;
the system of any preceding claims, adaptable and scalable for integration into various medical reporting environments beyond radiology; or
the system is tailored to the field of radiology, allowing radiologists to actively and seamlessly shape the behavior of the AI model.

### Advanced Dynamic User Interface for Radiology Reporting Systems

In certain embodiments, a method or system herein can overcome the limitations of known systems, in that it further comprises an advanced dynamic user interface (UI) incorporated into a Radiology Reporting System (RRS). In certain embodiment, the Radiology Reporting System can capture and manipulate API return calls from Language Learning Models (LLM), and further can integrate advanced RTF functionalities, real-time editing capabilities, drag and drop functionality (for example, a drag and drop sentence rearrangement), or any additional functionalities, e.g., within the “IMPRESSION” section of a radiology report.

In an exemplary embodiment, the features can include any of the following:

### Component 1: Advanced RTF Functions and Basic Formatting

System Architecture: The advanced RTF functions are made possible through a floating UI dock that integrates with the main reporting window.
Floating Dock Accessibility: The floating dock remains on-screen and is accessible regardless of scrolling activity, ensuring consistent access to text formatting tools.
Text Formatting Options: Includes options for text effects, e.g., bolding, underlining, italicizing, font-size manipulation, bulleting, numbering Use-Case: This feature can allow a radiologist to emphasize or delineate specific portions of text, contributing to more accurate and clearer reports.

### Component 2: Drag-and-Drop Sentences

System Architecture: This feature integrates with the primary reporting template and displays each sentence from the API return call as a bounded UI element.
Drag-and-Drop Functionality: Users can effortlessly rearrange these bounded sentences within the reporting template through a drag-and-drop interface.
Use-Case: This feature can be particularly useful for quickly organizing findings, comments, and interpretations in a logical or preferential manner.

### Component 3: One-Click Real-Time Editing

System Architecture: Unlike traditional systems where multiple clicks are required to enter an edit mode, in certain embodiments, this feature can allow real-time, one-click editing. [one-click]
Ease of Editing: Each bounded sentence or section can be instantly editable by merely clicking or highlighting a word or section.
Use-Case: This feature can save time and streamline the reporting process, especially when minor changes are required, reducing cognitive load on the healthcare professional and improving report quality.

### Component 4: Advanced “IMPRESSION” Section Functionality

System Architecture: The “IMPRESSION” section can be equipped with advanced user interface (UI) options, e.g., elements for a highly interactive experience.
Dynamic Elements: Deletion of sections through a simple delete icon; Sorting of sections using a sorting icon; Adding a new section with an add icon, automatically resorting the list; Drag-and-drop for rearranging sections, with automatic resorting; One-click real-time editing, as described above.
Use-Case: These features can bring unprecedented flexibility and control to a typically rigid aspect of radiology reporting, empowering radiologists to intuitively structure their impression section.

### Component 5:“Common Phrase” Insertion

System Architecture: An additional feature is the inclusion of a “common phrase” dropdown menu that appears upon clicking a “+” sign.
Phrase Functionality: This dropdown allows users to quickly pick a preset phrase to insert into their reports, while also allowing the freedom to add custom text.
Programmability: The dropdown list can be programmatically updated, offering adaptability to the user's requirements.
Use-Case: This reduces the repetitive nature of inserting common phrases, thus accelerating the report creation process.

In certain embodiments, the dynamic nature of this UI, particularly within the highly specific realm of radiology reporting, sets it apart from existing solutions, and confers many advantages. In particular, the methods and systems herein revolutionize radiology reporting by merging the power of LLMs with a highly dynamic and user-friendly interface. In certain embodiments, the inclusion of rich text formatting, advanced editing capabilities, and innovative features within the “IMPRESSION” section make the methods and systems herein not just a step forward but a leap in the field of radiology reporting. Further, built with an understanding of healthcare standards like FHIR, HL7, and DICOM, the methods and systems herein can ensure seamless integration with existing healthcare IT infrastructure. Given the architecture of the methods and systems herein, in certain embodiments have broad applicability and potential, in that they can be scaled or adapted for other medical reporting needs beyond radiology.

Thus, in various embodiments, the present technology is directed to: a radiology reporting system capable of providing a dynamic user interface, that optionally can include real-time text formatting and advanced editing capabilities.

And optionally, wherein any of the following are true of the system:
the UI allows for one-click, real-time editing of bounded sections or sentences;
the “IMPRESSION” section incorporates dynamic elements like sorting, adding, deleting, and rearranging of text sections;
a “common phrase” insertion functionality is provided, permitting a user to quickly select and insert commonly used phrases into the report; or
the system is compatible with healthcare IT standards, including but not limited to: FHIR, HL7, or DICOM.

### Specialized Radiology Reporting and Image Analysis Using Language Models

In certain embodiments, a method or system herein can include one or more specialized modules for various radiology and image analysis requirements, thus permitting real-time adaptability, robust interoperability, and secure auditing.

In certain embodiments, a method or system herein need not be limited to H17 or DICOM data formats, or specialized image data that require specific knowledge to interpret and use effectively; but rather, can provide an interoperability module that is configured to adopt other healthcare integration standards like Fast Healthcare Interoperability Resources (FHIR) standards, in addition to HL7 and DICOM standards.
Real-Time Adaptation: In certain embodiments, a method or system herein can learn from edge cases by employing one or generative AI, LLM based or machine learning models to identify patterns or anomalies in the incoming data, which can permit real-time adaptation to new data formats or updates to existing ones. In certain embodiments, a method or system herein can include a feedback loop for improvements—e.g., an automated mechanism that can learn from errors or oversights, continually refining the system's future capabilities.
Auditing and Accountability: In certain embodiments, a method or system herein can maintain immutable logs of all transactions and data translations, ensuring high levels of security and accountability; or it can track data modification with detailed records, complying with auditing standards and requirements; or it can perform periodic automated security audits, providing reports if necessary for regulatory compliance or quality control; or it can provide an alerting mechanism that can flag anomalies in the audit, to ensure system integrity.

In certain embodiments, a method or system herein can include a reverse translation module, that can take data generated from software applications, for example, radiology reports in JSON or other software-friendly formats, and convert it into healthcare system standard formats like HL7, FHIR, or DICOM for seamless integration back into the healthcare workflow. That is, in known methods, one would have to follow existing standards and programming methods to accomplish each of these tasks; specifically, with interface engines. However, with A.I. models trained to ingest the information as in certain methods and systems herein, it is now possible, with the present technology, to “leap-frog” and bypass those by creating models. For example, if a user receives an input from a healthcare system in HL7, it would be necessary to translate that input into developer code in the present system; that requires an interface such as FHIR. Conversely, an A.I. model according to a present embodiment herein, that understands HL7 (that is, it is trained to do so) can automatically convert the information coming in and translate it to something developer code can understand, e.g., JSON format.

Thus, in various embodiments, the present technology is directed to: a healthcare data translation, integration, and analysis system, including one or more specialized modules for technique identification, contrast usage, time and date of study, patient data, accession number/MRN, prior study information, facility details, or image analysis.

And optionally, wherein any of the following are true of the system:
the system is capable of real-time adaptation to handle new data formats or updates to existing formats;
the system includes robust auditing features for tracking changes, maintaining immutable logs, or performing security audits;
the system is at least partially interoperable with one or more existing healthcare systems through the adoption of healthcare integration standards, e.g., FHIR, in addition to HL7 and DICOM;
the system is capable of bidirectional data translation, including translating data from software-friendly formats back into healthcare system standard formats; and
the system has an image analysis module that utilizes Language Models to interpret and analyze image data, extracting relevant measurements and notes.

### Enhancing Radiological Reporting Through A.I.—Assisted Pre-Caching

In the realm of medical imaging, the advent of A.I. imaging analysis has ushered in an era of enhanced image recognition coupled with a profound understanding of the context enveloping these images. Such advancements hold paramount importance for myriad reasons. Specifically, in certain embodiments, the methods and systems herein delve into the nuanced interpretation of measurement images stemming from ultrasound evaluations and the associated worksheets crafted by ultrasound technologists or sonographers.

Currently, sonographers are tasked with embedding measurements directly onto the images. While some measurements are superimposed on the anatomical scans, a holistic summary image, encapsulating measurements of all pertinent anatomical structures, is typically generated. Radiologists rely on this summary image for dictating measurements. Complementing this, sonographers occasionally resort to handwritten worksheets. These not only mirror the measurements showcased in the summary image but also encompass pivotal notes pertinent to the specific study type.

The conventional radiologist workflow is multifaceted: it entails a meticulous review of all images, supplemented by the invaluable insights provided by sonographers, manifested either through ultrasound machine's summary images or through scanned worksheets consolidated under the patient's PACS profile. The subsequent step for the radiologist involves dictating these insights into their report, often echoing the sonographer's measurements. In certain embodiments herein, this repetitive transcribing process, although integral, has been subject to optimization through the power of A.I. and LLMs.

In certain embodiments herein, a method or system can include “report caching” in the context of radiological reporting. Report caching can employ A.I. and LLMs to pre-cache reports, ensuring expedited and efficient reporting, especially when immediate context from images is imperative.

To clarify using an ultrasound example: in certain embodiments, a method or system herein permits the AI/LLM system to prepopulate the radiology report with exact measurements even before the radiologist begins the case. Since sonographer measurements and notes are highly accurate and detailed, they are directly transcribed into reports as part of the established workflow. By pre-caching these reports, the system negates the need for real-time AI computations during the reporting phase. As a result, radiologists are presented with a pre-completed report the moment they open the case. This innovation significantly refines the reporting process, enhancing both the speed and computational efficiency.

The proposed method seeks to leverage the DICOM (Digital Imaging and Communications in Medicine) tag, specifically the “Study Description” identified by its tag reference (0008,1030). This tag indicates the type of study undertaken. Alternatively, the method can utilize the HL7 (Health Level Seven) protocol, extracting study-related data from the OBR (Observation Request) segment. Notably, the OBR-4 field, known as the Universal Service Identifier, is of interest. This field typically comprises a code alongside a description of the requested study or procedure.

It is worth noting that while the DICOM and HL7 methods serve as primary avenues for deciphering study types, the method remains adaptable to other viable techniques achieving the same objective.

Once the study type is identified, algorithmic filters are applied to segregate specific study categories, such as completed Ultrasound (US) studies. After isolating US studies, the algorithm then focuses on identifying scanned worksheets or summary measurement images through DICOM-based categorization.

An illustrative approach to this process is as follows:

### EXAMPLE 1

In the context of ultrasound imaging within DICOM (Digital Imaging and Communications in Medicine), specific data representations, such as the “Key Object Selection Document” or “Measurement Summary,” can be utilized to capture essential information. This data can be identified through several distinguishing methods:

1. Modality and SOP Class UID Identification:

The Modality is identified via its tag (0008,0060), commonly set to “US” for ultrasound. The SOP Class UID tag (0008,0016) for the Key Object Selection Document would correspond to UID 1.2.840.10008.5.1.4.1.1.88.59.

2. Content Sequence:

The Content Sequence (0040,A730) contains items that often incorporate the measurements and annotations. This can serve as an indicator for the summary image.

3. Concept Name Code Sequence:

The Concept Name Code Sequence tag (0040,A043) provides semantic information about the content item. It might denote an item as a “Measurement Summary” or a term of similar connotation.

4. Presence of Pixel Data:

The absence of the Pixel Data tag (7FE0,0010) can further distinguish between summary and other images.

After identifying the pertinent image or scanned worksheet for a specific study type (illustratively, US studies), the image, paired with a proprietary prompt, is processed by the AI/LLM to produce a response. Subsequent to obtaining the response, there are two predominant methods to prepopulate the report:

### Method 1: Prepopulating Reports in Advance

1—Extraction Process:

Upon capturing the ultrasound image (Key Object Selection Document), deploy a DICOM Listener/Processor to survey the storage area for new additions.

If a new DICOM object is detected, the Listener verifies if it corresponds to a Measurement Summary based on the previously mentioned criteria.

Extract pertinent measurement and annotation data from the DICOM header.

2—Report Formation:

Populate a template using the retrieved data, forming a preliminary report.

3—Storage of Preliminary Report:

Retain the preliminary report in a suitable system, such as the reporting system, information system, or PACS, linked with the study's UID.

4—Access by Radiologist:

As a radiologist initiates the study, the system automatically retrieves the associated prepopulated report, facilitating a streamlined evaluation.

### Method 2: UID-Based On-Demand Population

1—Extraction & Retention:

Similar to Method 1, upon detecting a Measurement Summary image, the system extracts relevant data.

Instead of immediate report generation, store this data in a transient storage area or local database within the reporting system, associating it with the study's UID.

2—Access by Radiologist:

As the radiologist accesses the study, an embedded process in the system checks for data tied to the study's UID.

3—Real-time Report Formation:

If corresponding data is located, the system instantaneously populates a report template with the extracted data, presenting this to the radiologist.

4—Fallback Mechanism:

In cases where matching data is absent or if there is an extraction error, the radiologist is either presented with a blank template or notified about the non-availability of prepopulated data.

The described methods and systems described above stand apart in uniqueness. Prior to the introduction of LLMs/AI in this capacity, radiology report pre-population based on summary images and scanned worksheets was not conceivable. Traditional approaches hinged upon extracting data elements directly from DICOM tags, necessitating intricate parsing. The inventive method and systems herein transcend such confines, pivoting from data parsing to direct image interpretation through LLMs/A.I.

Whereas previous attempts without the aid of LLMs/A.I. might necessitate real-time computations—leading to inefficiencies and report delivery lags—the approaches discussed herein introduce the principle of “report caching.” These processes ensure prompt availability of the requisite data for end-users, notably radiologists, streamlining the workflow and elevating the efficiency threshold.

It should be noted that known methods also have had to use structured reporting (SR) data to extract measurements when available. Not all ultrasound machines allow this, so it is a limitation, whereas the methods and systems disclosed herein do not rely on it. Traditional methodologies in radiology reporting have also utilized Structured Reporting (SR) data to standardize the extraction and documentation of diagnostic information. SR, by design, facilitates the systematic capture of data from imaging studies, promoting consistency and interoperability across different systems. This conventional approach often relies on SR data derived from DICOM (Digital Imaging and Communications in Medicine) standards, necessitating specific data parsing processes to extract and utilize this information

However, the innovative AI/LLM-driven workflow described herein introduces a flexible and efficient alternative to handling radiology data, including, but not limited to, SR data. While SR provides a structured format for data that enhances clarity and consistency in reports, its availability can vary, and its format can sometimes limit the agility of report generation. The present technology recognizes the value of SR data when available, and in certain embodiments, can integrate it into the reporting process. Nonetheless, the methods and systems herein are not solely dependent on this data format.

The AI/LLM-driven approach is designed to understand and interpret various data formats, including traditional SR data and unstructured or semi-structured inputs as well as OCR style image analysis. This versatility allows for the automatic conversion of incoming data—be it in HL7, DICOM SR, or other formats—into a structured and standardized output, such as JSON, without the need for traditional interface engines or extensive manual coding. In essence, this method can bypass the conventional parsing of SR data, providing a pathway to generating comprehensive, accurate, and standardized radiology reports. This integration capability ensures that the methods and systems herein remain adaptable and efficient, capable of leveraging SR data when available while not being hindered by its absence.

Thus, in various embodiments, the present technology is directed to: a method or system for report caching in a radiology application, wherein one or more summary images or scanned worksheets are interpreted directly through LLMs/AI, bypassing traditional data parsing from DICOM tags. That is, in certain embodiments, a method or system herein can include the steps of caching the report and a subsequent report generated by the same healthcare provider for a different patient or different procedure, wherein the image and additional patent data are interpreted directly through LLMs and A.I. without data parsing from a DICOM tag.

And optionally, wherein any of the following are true of the method:
the interpretation is achieved without real-time computations, reducing computational inefficiencies and report delivery lags;
the LLMs/AI discerns the study type through the “Study Description” DICOM tag, or by detecting study-related information from the OBR segment in HL7;
the report generation is triggered by the detection of particular DICOM criteria, including but not limited to “Key Object Selection Document,” Modality, SOP Class UID, Content Sequence, Concept Name Code Sequence, or the absence or presence of Pixel Data;
the report generation incorporates extracted data into specific sections or templates for preliminary reports, offering radiologists immediate access upon initiating the case evaluation;
the method or system utilizes an integrated DICOM Listener/Processor to monitor storage areas for new DICOM objects, triggering extraction and report generation processes upon detection;
the extracted data from the DICOM object is stored with a reference to the study's UID, facilitating quick, or even immediate, access for radiologists;
the method or system allows on-the-fly report generation by associating stored extracted data with a study's UID, presenting a prepopulated report upon radiologist access;
the method or system integrates fallback mechanisms to present radiologists with blank templates or notifications in the absence of prepopulated data; or
the method or system streamlines the reporting process, eliminates computational lags, or provides immediate, tailored reports to radiologists.

As can be seen through the present disclosure, the methods and systems herein exhibit numerous advantages over known methods and systems. In certain embodiments, a system herein includes a computer processor that is configured to provide the report without the need for additional revision or editing, including but not limited to human revision or editing. That is, unlike known systems, there is no need for further human intervention between translation of the healthcare professional's diagnosis and generation of the report.

In certain embodiments, a method herein includes a translating step that can be accomplished in a manner that satisfies an industry standard without the need for additional revision or editing.

As can be seen throughout the present disclosure, the ability to streamline the medical diagnosis process in such a manner is unprecedented, and without equal.

Although the present technology has been described in relation to embodiments thereof, these embodiments and examples are merely exemplary and not intended to be limiting. Many other variations and modifications and other uses will become apparent to those skilled in the art. The present technology should, therefore, not be limited by the specific disclosure herein, and can be embodied in other forms not explicitly described here, without departing from the spirit thereof.

We claim:

1. A method for generating a report based on a healthcare professional's review of a diagnostic image, the method comprising the steps of:
(a) obtaining, through a microphone, audio data in the form of speech in narrative form from the healthcare professional;
(b) generating, through a computer processor, a transcription of the speech in narrative form;
(c) translating, through a computer controlled voice recognition (VR) system, the speech in narrative form into speech suitable for a report that satisfies an industry standard; and
(d) providing a report that satisfies an industry standard.

2. The method of claim 1, wherein steps (a) through (d) are accomplished in parallel over 2 or more diagnostic images using the same computer processor.

3. The method of claim 1, wherein the diagnostic image is a DICOM image file.

4. A system for providing a report based on a healthcare professional's diagnosis, the system comprising:
(a) a user interface that displays a diagnostic image obtained from the body of a patient;
(b) a microphone configured to capture audio data in the form of speech in narrative form from the healthcare professional based on the healthcare professional's visual observation and diagnosis of the diagnostic image; and
(c) a computer processor configured to: (i) provide a transcription of the speech in voice recognition (VR) form based on the transcription speech in narrative form; (ii) associate the transcription of the speech in VR form with identifying information of the patient already stored in the system; and (iii) generate the report including the transcription of the speech in VR form and identifying information of the patient;
wherein the computer system is configured to conduct steps (a) through (c) for more than one patient in parallel or contemporaneously, while interacting with a single healthcare provider.

5. The system of claim 4, wherein the computer system includes parallel viewing software (PVS) and an integrated generative A.I., LLM-based application programming interface (API).

6. The system of claim 4, wherein the computer processor is configured to provide the report that satisfies an industry standard without the need for additional revision or editing.

7. The system of claim 4, further comprising A.I. model refinement or fine-tuning of one or more elements of the report.

8. The system of claim 4, further comprising a gamification element comprising one or more of the following: a feature wherein the user accumulates points through interactions; or a dashboard providing an overview of the user's progression.

9. The system of claim 4, further comprising one or more of the following: a dynamic user interface (UI); text formatting options for the report; drag and drop functionality; or an advanced user interface (UI) option.

10. The system of claim 4, wherein the system includes an interoperability module that is configured to adopt Fast Healthcare Interoperability Resources (FHIR) standards, in addition to HL7 and DICOM standards.

11. The system of claim 4, comprising an image analysis module that utilizes a Large Language Module (LLM).

12. A method of diagnosing a medical condition in a patient, the method comprising the steps of:
(a) scanning the body of the patient with an imaging apparatus;
(b) generating an image based on the scan;
(c) storing the image in a server along with additional patent data, wherein the image and the patient data are associated with each other in the server;
(d) obtaining a diagnosis of a medical condition from a medical professional, wherein the diagnosis is in the form of speech in narrative form from the healthcare professional; and
(e) translating the speech in narrative form into speech suitable for a report that satisfies an industry standard, through the application of machine learning to generate a report in written form, wherein the report also contains the additional patient data.

13. The method of claim 12, wherein the translating step is accomplished in a manner that satisfies an industry standard without the need for additional revision or editing.

14. The method of claim 12, further comprising the step of caching the report and a subsequent report generated by the same healthcare provider for a different patient or different procedure, wherein the image and additional patent data are interpreted directly through LLMs and A.I. without data parsing from a DICOM tag.

15. The method of claim 14, wherein image interpretation is achieved without real-time computations, reducing computational inefficiencies and report delivery lags.

16. The method of claim 14, wherein the LLMs/AI discerns the study type through the Study Description DICOM tag, or by detecting study-related information from the OBR segment in HL7.

17. The method of claim 14, wherein the report generation is triggered by the detection of particular DICOM criteria, including but not limited to Key Object Selection Document, Modality, SOP Class UID, Content Sequence, Concept Name Code Sequence, or the absence or presence of Pixel Data.

18. The method of claim 14, wherein report generation incorporates extracted data into specific sections or templates for preliminary reports, offering radiologists immediate access upon initiating the case evaluation.

19. The method of claim 14, further comprising an integrated DICOM Listener/Processor to monitor storage areas for new DICOM objects, triggering extraction and report generation processes upon detection.

20. The method of claim 19, wherein the extracted data from the DICOM object is stored with a reference to the study's UID, facilitating quick access for radiologists.

21. The method of claim 14, further comprising real-time report generation by associating stored extracted data with a study's UID, presenting a prepopulated report upon radiologist access.
